(19) United States
(12) Patent Application Publication
(10) Pub. No.: US 2022/0403339 A1
(43) Pub. Date: Dec. 22, 2022

(54) 3D HUMAN LIVER ORGAN MODEL CONSTRUCTING METHOD, 3D HUMAN LIVER ORGAN MODEL AND USE THEREOF

(71) Applicant: BEIJING DAXIANG BIOTECH CO., LTD, Beijing (CN)

(72) Inventors: Rongrong XIAO, Beijing (CN); Yu ZHOU, Beijing (CN)

(21) Appl. No.: 17/779,326

(22) PCT Filed: Jul. 15, 2020
(86) PCT No.: PCT/CN2020/102129

§ 371 Date: May 24, 2022

(30) Foreign Application Priority Data
May 15, 2020 (CN) 202010414270.2

(51) Int. Cl. C12N 5/071; G01N 33/50

(52) U.S. Cl. CPC C12N 5/0671; G01N 33/5082; C12N 2513/00; C12N 2509/00

(57) ABSTRACT

Provided is a 3D human liver organ model constructing method, comprising: preparing human primary liver cells, or mixed cells of same and liver non-parenchymal cells, or human liver cancer cell lines into a single cell suspension, and mixing the single cell suspension with a matrix material to obtain a mixed cell suspension; inoculating the mixed cell suspension into cultivation micropores of a 3D organ-on-a-chip, and carrying out cultivation at 37° C. to obtain a gelled 3D organ-on-a-chip; adding a culture medium into liquid storage holes of the organ-on-a-chip, and carrying out cultivation to obtain a 3D human liver organ model. Compared with other 2D human liver organ models, the constructed 3D human liver organ model has significantly enhanced response sensitivity to hepatotoxic drugs, and shows stronger hepatotoxic damage effect for reported hepatotoxic drugs. Compared with an animal model, the 3D human liver organ model can effectively eliminate the screening difference caused by species difference.

The application claims the priority of Chinese patent application No. 202010414270.2, filed on May 15, 2020, the contents of which are incorporated herein by reference in their entirety.

### TECHNICAL FIELD

The application relates to the technical field of biological tissue engineering, in particular to a 3D human liver organ model constructing method, a 3D human liver organ model and use thereof.

### BACKGROUND

At present, liver damage caused by drugs is a main reason for clinical loss of drugs, preventive warning and delisting after marketing. Therefore, more methods with good predictability are needed for evaluating a hepatotoxicity risk in a drug discovery process. Generally, American FDA and European Drug Administration require that new candidate drugs need to be subjected to toxicity assessment on rodents (such as mice) and non-rodents (such as dogs) before clinical trials.

Through investigation and comparison of targeted organ toxicity, it is found that the consistency rate of hepatotoxicity of drugs in animals and human beings in phase I clinical trials is very low, which is a main reason causing clinical hepatic failure and drug loss. In consideration of the scale of this challenge and its negative effect on the development of medical expenses and new treatment methods, there is a need to develop more methods that can replace animal models in the future with strong predictability and strong relevance to humans.

Hepatocytes are a target of in-vivo hepatotoxic damage, and human primary hepatocytes (PHHs) contain important components of drug metabolism and transport, and are considered as a gold standard for measuring liver functions. Under the condition of in-vitro traditional 2D PHH plate monolayer culture, PHHs quickly lose liver phenotype, liver metabolism function and cell viability, and are not suitable for hepatotoxicity test. Compared with a conventional 2D culture method, the 3D liver cell culture technology can enhance the liver phenotype, the metabolic activity and the cell culture stability. The 3D hepatocyte culture technology is an emerging tool which can be used for evaluating a hepatotoxicity mechanism and screening hepatotoxicity drugs. The existing 3D liver culture technology mainly includes a 3D culture method based on hydrogel and a synthetic scaffold and a matrix-free 3D hepatic globule.

In the process of realizing the embodiments of the disclosure, at least the following problems are found in the related technology: an artificially synthesized scaffold material is generally different from an in-vivo ECM environment and is not bionic enough. The existing mainstream matrix-free hepatic globule technology needs to form a globule in advance for a week, the growth is not easy to control, and cell necrosis caused by cell hypoxia is easy to occur in the central area of the hepatic globule. A 3D culture technology based on in-vivo synthesized natural hydrogel is the most bionic 3D culture technology, but the technology is not applied to preclinical safety evaluation of hepatotoxic drugs, and the response sensitivity of the existing 3D human liver organ model to hepatotoxic drugs needs to be further improved.

### SUMMARY

In order to have a basic understanding of some aspects of the disclosed embodiments, a simple summary is given below. The summary is not a general review, nor a protection range to determine key/important constituent elements or depict these embodiments, but is a foreword in the following detailed description.

The embodiments of the disclosure provide a 3D human liver organ model constructing method, a 3D human liver organ model and use thereof, so as to solve the technical problem that the response sensitivity of the existing 3D human liver organ model to hepatotoxic drugs needs to be further improved.

In some embodiments, the 3D human liver organ model constructing method includes the following steps:

preparing human primary liver cells, or mixed cells of the human primary liver cells and liver non-parenchymal cells, or human liver cancer cell lines into a single cell suspension;

mixing the single cell suspension with a matrix material to obtain a mixed cell suspension;

inoculating cultivation micropores of a 3D organ-on-a-chip with the mixed cell suspension, and carrying out cultivation at 37° C. to obtain a gelled 3D organ-on-a-chip: wherein in each cultivation micropore, an inoculation volume of the mixed cell suspension is 6-10 μL, the number of inoculated cells of the human primary liver cells or the mixed cells of the human primary liver cells and the liver non-parenchymal cells is 2500-25000, and the number of inoculated cells of the human liver cancer cell lines is 500-10000; and

adding a culture medium into liquid storage holes of the gelled 3D organ-on-a-chip, and carrying out cultivation at 37° C. to obtain a 3D human liver organ model.

In some embodiments, the 3D human liver organ model is constructed by adopting the above 3D human liver organ model constructing method.

In some embodiments, use of the 3D human liver organ model for screening of hepatotoxic drugs is provided.

The 3D human liver organ model constructing method, the 3D human liver organ model and the use thereof provided by the embodiments of the disclosure can achieve the following technical effects:

according to the 3D human liver organ model constructing method disclosed by the embodiments of the disclosure, by regulating and controlling the inoculation volume of the mixed cell suspension in each cultivation micropore and the number of inoculated cells, cells in the constructed 3D human liver organ model grow in an agglomeration form, the globular shapes of the cells are fuller, and more and longer pseudopodia extend out. The liver function of the liver organ model is subjected to characterization, and the result shows that the 3D human liver organ model is more bionic. The response sensitivity of the constructed 3D human liver organ model to the hepatotoxic drugs is significantly enhanced, compared with other 2D and 3D human liver organ models, the constructed 3D human liver organ model shows stronger hepatotoxic damage effect for the reported hepatotoxic drugs, and compared with an animal model, the 3D human liver organ model can effectively eliminate the screening difference caused by species difference. Therefore, the 3D human liver organ model can detect more drugs with a hepatotoxic damage effect in vitro, and is closer to that of clinical reports.

The above overall description and the description in the following are only exemplary and interpretive, and are not used to limit the present application.

### BRIEF DESCRIPTION OF THE DRAWINGS

One or more embodiments are illustratively illustrated by the accompanying drawings corresponding thereto, these illustrative descriptions and accompanying drawings do not constitute limitations to the embodiments, elements with the same reference numeral in the accompanying drawings are shown as similar elements, and the accompanying drawings do not constitute proportional limitations, and wherein:

FIG. 1 is a block diagram of a 3D human liver organ model constructing method provided by the embodiments of the disclosure:

FIG. 2 is a cellular morphology diagram of a constructed 3D human liver organ model provided by the embodiments of the disclosure;

FIG. 3 is a cellular morphology diagram of a 2D human liver organ comparison model constructed in comparative example 1;

FIG. 4 is a cell growth characterization curve graph of a constructed 3D human liver organ model provided by the embodiments of the disclosure;

FIG. 5 is a cell growth characterization curve graph of the 2D human liver organ comparison model constructed in comparative example 1;

FIG. 6 is a comparison histogram of albumin secretion detection results of the constructed 3D human liver organ model provided by the embodiments of the disclosure and the 2D human liver organ comparison model constructed in comparative example 1;

FIG. 7 is a mRNA quantitative characterization diagram of metabolic enzymes of the constructed 3D human liver organ model provided by the embodiments of the disclosure and the 2D human liver organ comparison model constructed in comparative example 1;

FIG. 8 is a graph showing hepatotoxic drug screening results of the constructed 3D human liver organ model provided by the embodiments of the disclosure and the 2D human liver organ comparison model constructed in comparative example 1:

FIG. 9 is a cellular morphology diagram of another constructed 3D human liver organ model provided by the embodiments of the disclosure:

FIG. 10 is a cellular morphology diagram of another constructed 3D human liver organ model provided by the embodiments of the disclosure;

FIG. 11 is a cellular morphology diagram of another 2D human liver organ comparison model constructed in comparative example 2;

FIG. 12 is a cellular morphology diagram of a 3D human liver organ comparison model constructed in comparative example 3;

FIG. 13 is a cell growth characterization curve graph of another constructed 3D human liver organ model provided by the embodiments of the disclosure, the 2D human liver organ comparison model constructed in comparative example 2 and the 3D human liver organ comparison model constructed in comparative example 3;

FIG. 14 is a comparison histogram of albumin secretion detection results of another constructed 3D human liver organ model provided by the embodiments of the disclosure and the 2D human liver organ comparison model constructed in comparative example 2;

FIG. 15 is a graph showing hepatotoxic drug screening results of another constructed 3D human liver organ model provided by the embodiments of the disclosure and the 2D human liver organ comparison model constructed in comparative example 2:

FIG. 16 is a representative result diagram of another constructed 3D human liver organ model provided by the embodiments of the present disclosure for screening of clinically reported drugs with low hepatotoxicity:

FIG. 17 is a representative result diagram of another constructed 3D human liver organ model provided by the embodiments of the present disclosure for screening of clinically reported drugs with no hepatotoxicity:

FIG. 18 is a structural schematic diagram of a 3D organ-on-a-chip adopted by the 3D human liver organ model constructing method disclosed by the embodiments of the disclosure; and

FIG. 19 is a stepped section structural schematic diagram of the 3D organ-on-a-chip shown in FIG. 18.

### REFERENCE SIGNS

10, liquid storage layer: 100, liquid containing tank; 101, gap; 102, frame; 11, liquid storage through hole; 20, 3D culture layer; 21, cultivation micropore; and 30, bottom plate layer.

### DETAILED DESCRIPTION

In order to understand the characteristics and the technical content of the embodiments of the disclosure in detail, the implementation of the embodiments of the disclosure is elaborated in detail in combination with the accompanying drawings, and the accompanying drawings are only used for reference description and are not used for limiting the embodiments of the disclosure. In the following technical description, for convenient interpretation, a plurality of details are used to provide sufficient understanding of the disclosed embodiments. However, one or more embodiments may still be implemented without these details. In other cases, in order to simplify accompanying drawings, known structures and devices can be shown in a simplified manner.

It should be noted that the embodiments in the embodiments of the disclosure and the features in the embodiments can be mutually combined without conflicts.

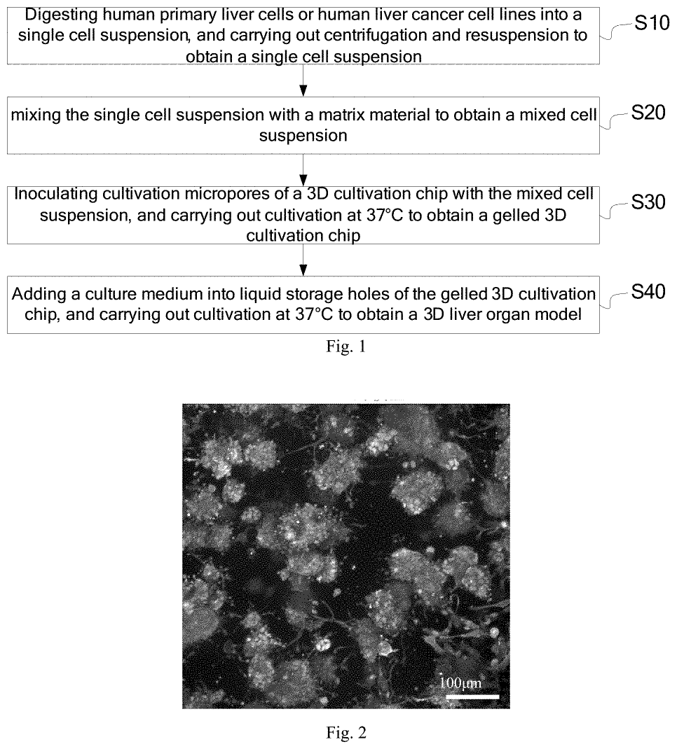

In combination with FIG. 1, the embodiments of the disclosure provide a 3D human liver organ model constructing method, including the following steps:

S10, preparing human primary liver cells, or mixed cells of the human primary liver cells and liver non-parenchymal cells, or human liver cancer cell lines into a single cell suspension:

S20, mixing the single cell suspension prepared in the step S10 with a matrix material to obtain a mixed cell suspension:

S30, inoculating cultivation micropores of a 3D organ-on-a-chip with the mixed cell suspension obtained in the step S20, and carrying out cultivation at 37° C. to obtain a gelled 3D organ-on-a-chip; wherein in each cultivation micropore, an inoculation volume of the mixed cell suspension is 6-10 μL, the number of inoculated cells of the human primary liver cells is 2500-25000, and the number of inoculated cells of the human liver cancer cell lines is 500-10000; and S40, adding a culture medium into liquid storage holes of the gelled 3D organ-on-a-chip, and carrying out cultivation at 37° C. to obtain a 3D human liver organ model.

According to the 3D human liver organ model constructing method disclosed by the embodiments of the disclosure, by regulating and controlling the inoculation volume of the mixed cell suspension in each cultivation micropore and the number of inoculated cells, cells in the constructed 3D human liver organ model grow in an agglomeration form, the globular shapes of the cells are fuller, and more and longer pseudopodia extend out. The liver function of the liver organ model is subjected to characterization, and the result shows that the 3D human liver organ model is more bionic. The response sensitivity of the constructed 3D human liver organ model to the hepatotoxic drugs is significantly enhanced, compared with other 2D human liver organ models, the constructed 3D human liver organ model shows stronger hepatotoxic damage effect for the reported hepatotoxic drugs, and compared with an animal model, the 3D human liver organ model can effectively eliminate the screening difference caused by species difference. Therefore, the 3D human liver organ model can detect more drugs with a hepatotoxic damage effect in vitro, and is closer to that of clinical reports.

The 3D human liver organ model system constructed in the embodiments of the disclosure can realize an in-vitro model combining three-dimensional bionic liver organs and high-throughput hepatotoxic drug screening, and is used for in-vitro hepatotoxic compound screening and related research. According to the liver organ model, liver function indexes such as albumin expression and urea secretion are remarkably improved, mRNA expression of main metabolic enzymes is also remarkably enhanced, and the liver organ model is more sensitive to response to hepatotoxic drugs.

In the embodiments of the disclosure, in the adopted 3D organ-on-a-chip, cultivation holes include cultivation micropores and liquid storage holes communicating with one another and coaxially arranged, and a hole diameter of the liquid storage hole is larger than that of the cultivation micropore. The design of large and small holes makes the operations of 3D cell inoculation and medium change more convenient, and accurate positioning of imaging easier.

In the embodiments of the disclosure, human primary liver cells, or mixed cells of the human primary liver cells and other liver non-parenchymal cells, or human liver cancer cell lines are adopted as model cells to construct the liver organ model, and there cells can be obtained through amplification by an existing amplification method and can also be obtained through purchase.

Optionally, the human primary liver cells and the liver non-parenchymal cells are mixed according to an in-vivo real proportion in the mixed cells of the human primary liver cells and the liver non-parenchymal cells. Optionally, the liver non-parenchymal cells are one or more of kuffer cells, vascular endothelial cells and astrocytes.

In the step S10 of the embodiments of the disclosure, a preparation method of the single cell suspension adopts the existing conventional means. The density of the single cell suspension can be determined according to a mixing proportion of the single cell suspension and the matrix material in the mixed cell suspension in the step S20, and the inoculation volume and the number of inoculated cells in each cultivation micropore in the step S30.

In some embodiments, in the step S10, human primary liver cells or mixed cells of the human primary liver cells and liver non-parenchymal cells are digested into a single cell suspension, and centrifugation and resuspension are performed to obtain a single cell suspension with a density of (4.45-44.5)×10⁶ cell/mL. Optionally, a single cell suspension with a density of (5-30)×10′ cell/mL is prepared. Optionally, a single cell suspension with a density of (6-20)×10 cell/mL is prepared. Optionally, a single cell suspension with a density of (7-10)×10⁶ cell/mL is prepared.

Optionally, in the step S10, human primary liver cells or mixed cells of the human primary liver cells and liver non-parenchymal cells are digested into a single cell suspension, and centrifugation and resuspension are performed to obtain a single cell suspension with a density of 8.9×10 cell/mL.

In some embodiments, in the step S10, human liver cancer cell lines hepG2 are digested into a single cell suspension, and centrifugation and resuspension are performed to obtain a single cell suspension with a density of (0.89-8.9)×10⁶ cell/mL. Optionally, a single cell suspension with a density of (1-8)×10⁶ cell/mL is prepared. Optionally, a single cell suspension with a density of (2-6)×10⁶ cell/mL is prepared. Optionally, a single cell suspension with a density of (3-4)×10⁶ cell/mL is prepared.

Optionally, the human liver cancer cell lines hepG2 are digested into a single cell suspension, and centrifugation and resuspension are performed to obtain a single cell suspension with a density of 3.56×10⁶ cell/mL.

In some embodiments, in the step S10, 0.25 (vt.)% pancreatin is utilized to digest 2D cultured human liver cancer cell lines in a growing period into a single cell suspension.

In some embodiments, in the step S10, human primary liver cells preserved by liquid nitrogen is resuscitated, and the human primary liver cells are diluted into a single cell suspension.

In some embodiments, in the step S20, a pH value of the mixed cell suspension is 6.5-7.5. Optionally, the pH value of the mixed cell suspension is 6.5-6.7. The pH value of the mixed cell suspension is adjusted to a suitable range by controlling a pH value of the matrix material.

In some embodiments, in the step S20, the matrix material includes, but is not limited to, one or more of collagen, matrigel and hydrogel of a natural source; when several matrix materials are adopted, a mixing proportion is not limited.

Optionally, the hydrogel of the natural source includes, but is not limited to, agar, chitosan, alginate, gelatin, fibroinectin, laminin, and the like.

Optionally, the matrix material is a mixed matrix material including collagen and matrigel; wherein a volume ratio of the collagen to the matrigel is 1:(0.5-2). Optionally, the volume ratio of the collagen to the matrigel is 1:(0.8-1).

In some embodiments, a preparation method of the mixed cell suspension in the step S20 includes: adding an alkaline solution into a matrix material, adjusting the pH value of the matrix material to 6.5-7.5, and then mixing the single cell suspension with the matrix material to obtain the mixed cell suspension.

Optionally, the alkaline solution can be one or a mixed solution of more of NaOH, NaHCO₃, and NaOH and NaHCO₃.

Optionally, a mixing volume ratio of the single cell suspension to the matrix material is 5.6:2.4, wherein the concentration of the matrix material is 5 mg/mL.

In some embodiments, in the step S30, in each cultivation micropore, the inoculation volume of the mixed cell suspension is 6-9 μL. Optionally, in each cultivation micropore, the inoculation volume of the mixed cell suspension is 7-8 μL.

In some embodiments, in the step S30, in each cultivation micropore, the number of inoculated cells of the human primary liver cells is 5000-15000. Optionally, the number of inoculated cells of the human primary liver cells is 8000-12000.

In some embodiments, in the step S30, in each cultivation micropore, the number of inoculated cells of the human liver cancer cell lines is 500-5000. Optionally, the number of inoculated cells of the human liver cancer cell lines is 500-3000.

In some embodiments, in the step S40, after the culture medium is added into the liquid storage holes of the gelled 3D organ-on-a-chip, a step that a buffer solution is added into an anti-evaporation structure of the gelled 3D organ-on-a-chip is further included. The humidity of the 3D organ-on-a-chip is increased, the evaporation degree is reduced, the consumption of a cell culture medium is reduced, and the performance of the constructed liver organ model is improved.

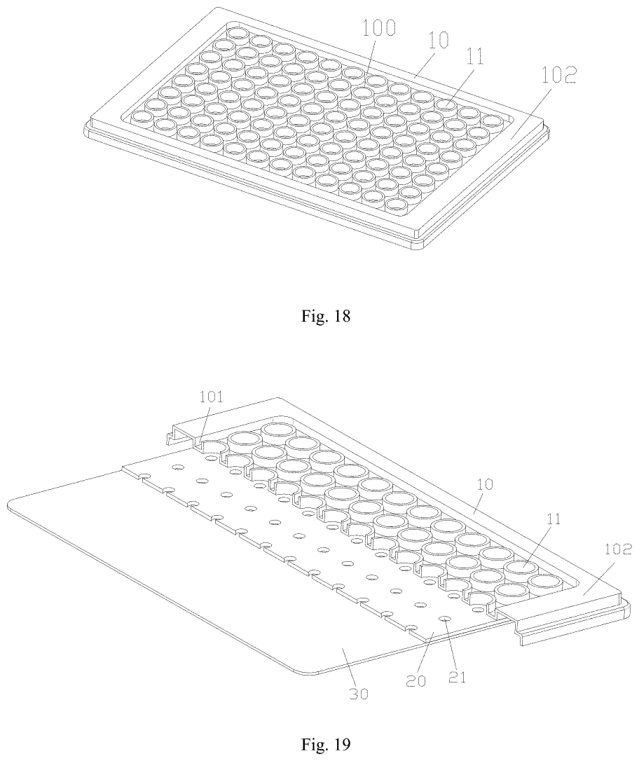

In the embodiments of the disclosure, in the adopted 3D organ-on-a-chip, cultivation holes include cultivation micropores and liquid storage holes communicating with one another and coaxially arranged, and a hole diameter of the liquid storage hole is larger than that of the cultivation micropore. The cultivation chip is also provided with an anti-evaporation structure, for example, a gap 101 shown in a 3D organ-on-a-chip shown in FIG. 18 and FIG. 19 is an anti-evaporation structure, that is, a groove arranged around a cultivation hole area is used as an anti-evaporation channel, and the anti-evaporation channel is filled with a buffer solution. As shown in FIG. 18 and FIG. 19, the 3D organ-on-a-chip includes a liquid storage layer 10, a 3D culture layer 20 and a bottom plate layer 30 which are sequentially arranged in a layered manner. The liquid storage layer 10 is provided with a plurality of liquid storage through holes 11, and the liquid storage through holes 11 are used for storing a culture solution; the 3D culture layer 20 is provided with a plurality of cultivation micropores 21, and the cultivation micropores 21 are used for 3D cell culture; and the liquid storage through holes 11 are in one-to-one correspondence with the cultivation micropores 21. Each liquid storage through hole 11 is a liquid storage cylindrical hole, and a frame 102 is arranged on the liquid storage layer around a liquid storage through hole area in a surrounding mode to form a liquid containing tank 100. The gap 101 is formed between the liquid storage through hole area and the inner side wall (namely, the inner peripheral wall of the liquid containing tank) of the frame 102. The gap 101 is annular, can be used as a solution channel, provides a channel for a PBS buffer solution, and is used for increasing the humidity of the whole pore plate and reducing the evaporation degree. The 3D organ-on-a-chip capable of being adopted in the embodiments of the disclosure can specifically refer to the content disclosed in the Chinese utility model patent CN209989412U, entitled a 3D high-throughput organ-on-a-microchip.

Optionally, the buffer solution is a PBS buffer solution (a phosphate buffer solution).

The embodiments of the disclosure further provide a 3D human liver organ model which is constructed by adopting the above 3D human liver organ model constructing method.

The embodiments of the disclosure further provide use of the 3D human liver organ model for screening of hepatotoxic drugs.

In the embodiments of the disclosure, in screening of the hepatotoxic drugs, the drugs include nefazodone, sunitinib, tamoxifen, chlorpromazine, bosentan, rosiglitazone, simvastatin, celecoxib, tetracycline, glafenine, sulindac, amitriptyline, verapamil, tolbutamide and paracetanol. The results of large-scale hepatotoxic drug screening are obtained from 122 marketed drugs.

Specific embodiments are given below to illustrate the 3D human liver organ model constructing method provided by the embodiments of the disclosure.

### Embodiment 1

A 3D human liver organ model constructing method, including the following steps:

S11, digesting 2D cultured human liver cancer cell lines hepG2 in a growth period into a single cell suspension by using 0.25 (vt.)% pancreatin, and carrying out centrifugation and resuspension to obtain a single cell suspension with a density of (3.35-8.9)×10⁶ cell/mL:

S21, adding the single cell suspension and a matrix material into a 1.5 ml EP tube according to a volume ratio of the single cell suspension to the matrix material being 5.6:2.4, and conducting uniform mixing through pipetting by a pipette to obtain a mixed cell suspension; wherein the matrix material is a mixed matrix material of collagen and matrigel, a volume ratio of the collagen to the matrigel is 1:1, the concentration of the collagen is 2.5 mg/mL, and the concentration of the matrigel is 5 mg/mL.

S31, quickly transferring the mixed cell suspension at high throughput by using a pipette to inoculate cultivation micropores of a 3D organ-on-a-chip, then placing the 3D organ-on-a-chip inoculated with the mixed cell suspension in a cell incubator, carrying out cultivation at 37° C. for 10 min while ensuring that collagen can be gelled well, and taking out the 3D organ-on-a-chip to obtain a gelled 3D organ-on-a-chip; wherein in each cultivation micropore, the inoculation volume of the mixed cell suspension is 8 μL, and the number of inoculated cells of the human liver cancer cell lines is 2000-10000.

S41, adding a culture medium into liquid storage holes of the gelled 3D organ-on-a-chip obtained in the step S31, and then adding a PBS buffer solution into an anti-evaporation channel of the gelled 3D organ-on-a-chip. And carrying out cultivation at 37° C. to obtain a 3D human liver organ model.

In this embodiment 1, a 3D human liver organ model I is constructed. Wherein different 3D human liver organ models I are constructed according to different numbers of inoculated cells in each cultivation micropore in the step S31.

A 3D human liver organ model I-1 is constructed according to the fact that the number of cells in each cultivation micropore in the step S31 is 2000.

A 3D human liver organ model I-2 is constructed according to the fact that the number of cells in each cultivation micropore in the step S31 is 3000.

A 3D human liver organ model I-3 is constructed according to the fact that the number of cells in each cultivation micropore in the step S31 is 5000.

In this embodiment 1. F-actin and nuclear dyeing characterization is carried out on the 3D human liver organ model I. As shown in a cellular morphology diagram of a 3D human liver organ model I-1 in FIG. 2, it can be seen that liver cells in the 3D human liver organ model I grow in a spherical shape on a 3D platform, and elongate pseudopodia. As shown in a cell growth characterization curve graph of a 3D human liver organ model I in FIG. 4, it can be seen that on the 3D human liver organ model I, cells can stably grow and keep vitality for 12 days, and the 3D human liver organ model I is suitable for chronic toxicity screening. Wherein in FIG. 4, “-▪-” is a cell growth curve of the 3D human liver organ model I-1, “-▴-” is a cell growth curve of the 3D human liver organ model I-2, and “-▾-” is a cell growth curve of the 3D human liver organ model I-3.

In this embodiment 1, the 3D human liver organ model I is subjected to liver function characterization. Wherein albumin secretion detection is respectively carried out on the 3D human liver organ model I-1 which is cultured for 5 days and 7 days, as shown in FIG. 6. Wherein each gray bar represents the 3D human liver organ model I-1 in this embodiment 1: each black bar represents a 2D liver organ comparation model I in comparative example 1. The results show that compared with a 2D liver model, the albumin secretion amount of the 3D model I-1 on day 5 is 2.2 times that of a 2D comparison model IL the albumin secretion amount of the 3D model I-1 on day 7 is 1.8 times that of the 2D comparison model I, and the albumin secretion amount of the 3D liver model is significantly increased (P<0.005).

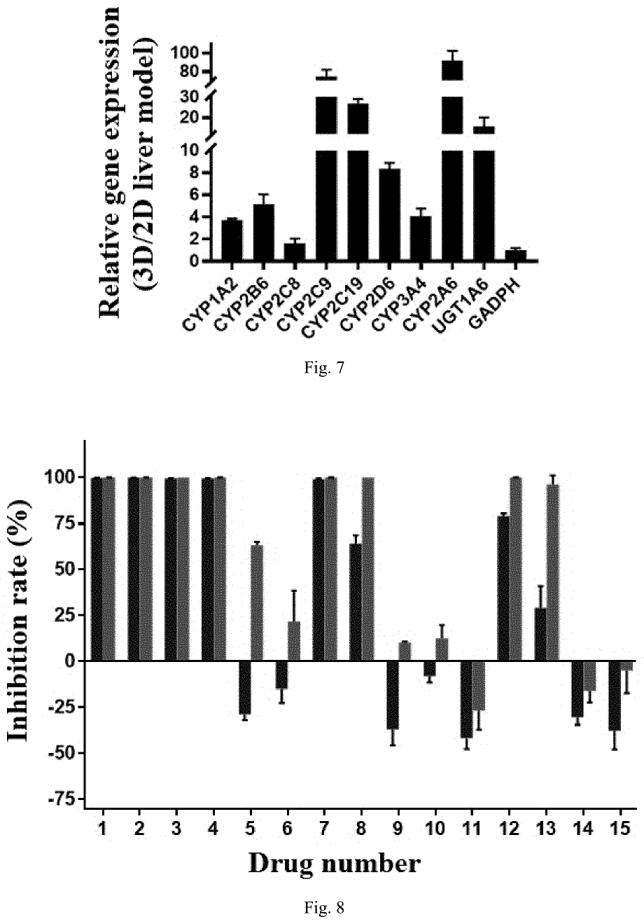

As shown in FIG. 7, a mRNA quantitative characterization diagram of metabolic enzymes of the 3D human liver organ model I-1 cultured for 7 days relative to the 2D comparison model I in comparative example 1 is shown. mRNA of enzymes expressed in a liver model cell line is quantified (a same batch of cells, one part of a cell suspension digested by a 2D plate is used for 2D mRNA extraction, one part of the cell suspension is used for inoculation to form a 3D liver organ model, and the mRNA is extracted after 3D-2000 cells are seeded for 7 days), and the result shows that the mRNA expression quantity of the majority of the metabolic enzymes on the 3D liver model is significantly increased compared with the 2D liver model, and the increase quantity can be up to 100 times.

As shown in FIG. 8, a graph showing hepatotoxic drug screening results of a 3D human liver organ model I-1 (gray bars in the figure) in this embodiment 1 and a 2D liver organ comparison model I (black bars in the figure) in comparative example 1 is shown. Wherein drugs represented by a drug number of 1-15 are sequentially as follows: nefazodone, sunitinib, tamoxifen, chlorpromazine, bosentan, rosiglitazone, simvastatin, celecoxib, tetracycline, glafenine, sulindac, amitriptyline, verapamil, tolbutamide and paracetamol. A hepatotoxic drug screening result shows that compared with a drug screening result of a 2D cell line liver organ comparison model, more hepatotoxic drugs (such as numbered 5, 6, 8, 12 and 13) can be detected to be have stronger hepatotoxicity inhibition on a 3D cell line liver organ model. On the whole, the response sensitivity of the 3D human liver organ model constructed in this embodiment 1 to hepatotoxic drugs is remarkably enhanced. It can be known that the drugs with the numbers of 5, 6, 8, 12 and 13 are clinically confirmed to be low-hepatotoxicity drugs, and it can be seen that the hepatotoxic drug screening result of the 3D human liver organ model I in this embodiment 1 has very high clinical guiding significance.

### Embodiment 2

A 3D human liver organ model constructing method, including the following steps: S12, resuscitating human primary liver cells preserved by liquid nitrogen, diluting the human primary liver cells into a single cell suspension, and carrying out centrifugation and resuspension to obtain a single cell suspension with a density of (4.45-44.5)×10⁶ cell/mL. S22, adding the single cell suspension and a matrix material into a 1.5 ml EP tube according to a volume ratio of the single cell suspension to the matrix material being 5.6:2.4, and conducting uniform mixing through pipetting by a pipette to obtain a mixed cell suspension: wherein the matrix material is a mixed matrix material of collagen and matrigel, a volume ratio of the collagen to the matrigel is 1:1, the concentration of the collagen is 2.5 mg/mL, and the concentration of the matrigel is 5 mg/mL.

S32, quickly transferring the mixed cell suspension at high throughput by using a multichannel pipette to inoculate cultivation micropores of a 3D organ-on-a-chip, then placing the 3D organ-on-a-chip inoculated with the mixed cell suspension in a cell incubator, carrying out cultivation at 37° C. for 10 min while ensuring that collagen can be gelled well, and taking out the 3D organ-on-a-chip to obtain a gelled 3D organ-on-a-chip; wherein in each cultivation micropore, the inoculation volume of the mixed cell suspension is 8 μL, and the number of inoculated cells of the human primary liver cells is 2500-25000.

S42, adding a culture medium into liquid storage holes of the gelled 3D organ-on-a-chip obtained in the step S32, and then adding a PBS buffer solution into an anti-evaporation channel of the gelled 3D organ-on-a-chip. And carrying out cultivation at 37° C. to obtain a 3D human liver organ model.

A 3D human liver organ model II is constructed in this embodiment 2. Wherein different 3D human liver organ models II are constructed according to different numbers of inoculated cells in each cultivation micropore in the step S32.

A 3D human liver organ model II-1 is constructed according to the fact that the number of inoculated cells in each cultivation micropore in the step S32 is 3000.

A 3D human liver organ model II-2 is constructed according to the fact that the number of inoculated cells in each cultivation micropore in the step S32 is 5000.

A 3D human liver organ model II-3 is constructed according to the fact that the number of inoculated cells in each cultivation micropore in the step S32 is 15000.

A 3D human liver organ model II-4 is constructed according to the fact that the number of inoculated cells in each cultivation micropore in the step S32 is 25000.

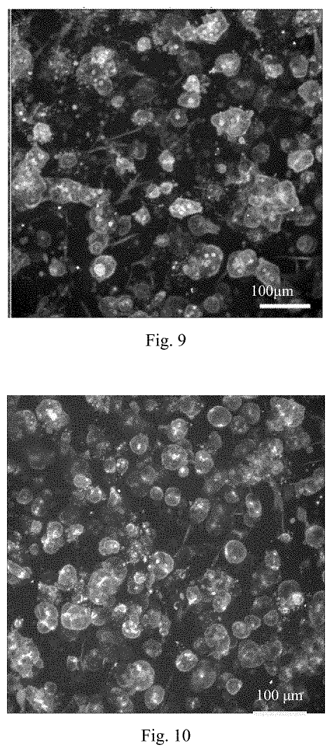

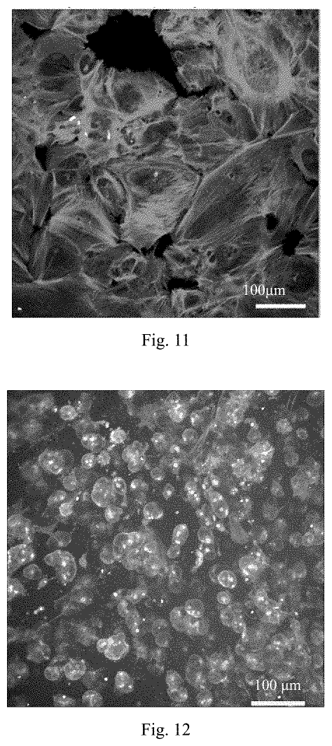

In this embodiment 2, F-actin and nuclear dyeing characterization is carried out on the 3D human liver organ model II. As shown in a cellular morphology diagram of a 3D human liver organ model II-2 in FIG. 9, and a cellular morphology diagram of a 3D human liver organ model II-4 in FIG. 10, it can be seen that the liver cells in the 3D human liver organ model II grow in a spherical shape on a 3D platform, and elongate pseudopodia.

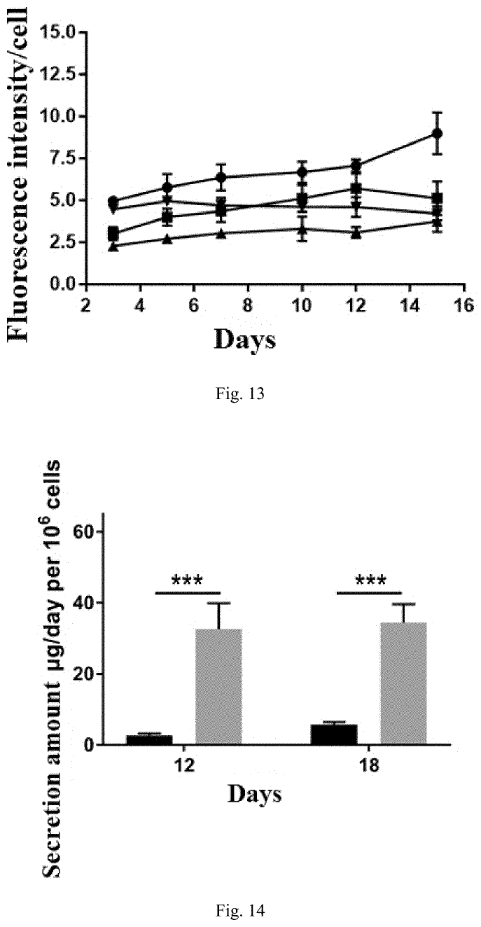

As shown in a cell growth characterization curve of a 3D human liver organ model II-2 represented by “-●-” in FIG. 13, it can be seen that on the 3D human liver organ model II-2, cells can stably grow and keep vitality for 15 days, and the 3D human liver organ model II-2 is suitable for chronic toxicity screening. As shown in a cell growth characterization curve of a 3D human liver organ model II-4 represented by “-▪-” in FIG. 13, it can be seen that on the 3D human liver organ model II-4, cells can stably grow and keep vitality for 12 days, and the 3D human liver organ model II-4 is also suitable for chronic toxicity screening. A cell growth characterization curve of a 2D human liver organ comparison model II in comparative example 2 represented by “-▾-” as shown in FIG. 13 shows that on the 2D human liver organ comparison model II, cells can only grow for 5 days at most, the vitality of the cells is in a decline state, and the overall vitality of the cells on the 2D human liver organ comparison model H is obviously lower than the vitalities of the cells on the 3D human liver organ model II-2 and the 3D human liver organ model II-4. A cell growth characterization curve of a 3D human liver organ comparison model III in comparative example 3 represented by “-▴-” as shown in FIG. 13 shows that although cells are always in a vitality rising state, its overall vitality is obviously lower than those in the 3D human liver organ model II-2 and the 3D human liver organ model II-4 in this embodiment 2, and it can be known that in the 3D human liver organ model, the vitality of a single cell is reduced along with increase of the number of inoculated cells.

In this embodiment 2, the 3D human liver organ model II is subjected to liver function characterization. Wherein albumin secretion detection is respectively carried out on the 3D human liver organ model II-2 cultured for 12 days and 18 days, as shown in FIG. 14. Wherein each gray bar represents the 3D human liver organ model II-2 in this embodiment 2; each black bar represents a 2D liver organ comparation model II in comparative example 2. The result shows that compared with the 2D comparation model II, the albumin secretion amount of the 3D model II-2 on day 12 is 12 times that of the 2D comparation model II, the albumin secretion amount of the 3D model II-2 on day 18 is 6 times that of the 2D comparation model II, and the albumin secretion amount is remarkably increased (P<0.005).

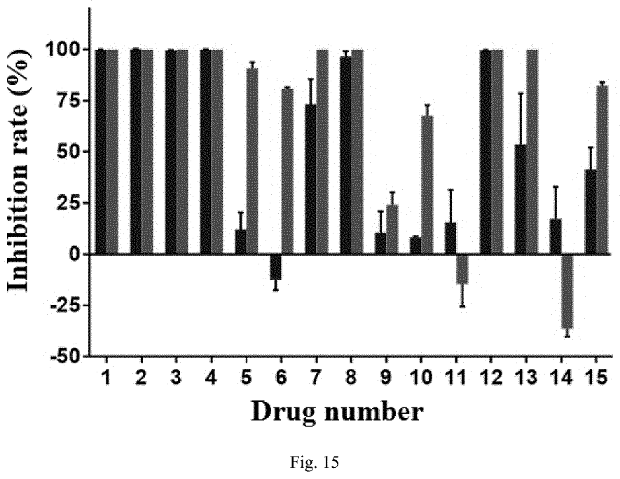

As shown in FIG. 15, a graph showing hepatotoxic drug screening results of a 3D human liver organ model II-2 (gray bars in the figure) in this embodiment 2 and a 2D liver organ comparison model II (black bars in the figure) in comparative example 2 is shown. Wherein drugs represented by a drug number of 1-15 are sequentially as follows: nefazodone, sunitinib, tamoxifen, chlorpromazine, bosentan, rosiglitazone, simvastatin, celecoxib, tetracycline, glafenine, sulindac, amitriptyline, verapamil, tolbutamide and paracetamol. A hepatotoxic drug screening result shows that compared with a drug screening result of a 2D cell line liver organ comparison model, more hepatotoxic drugs (such as numbered 5, 6, 7, 9, 10, 13 and 15) can be detected to be have stronger hepatotoxicity inhibition on the 3D cell line liver organ model. On the whole, the response sensitivity of the 3D human liver organ model constructed in this embodiment 2 to hepatotoxic drugs is remarkably enhanced. It can be known that the drugs with the numbers of 5, 6, 7, 9, 10, 13 and 15 are clinically confirmed to be low-hepatotoxicity drugs, and it can be seen that the hepatotoxic drug screening result of the 3D human liver organ model II in this embodiment 2 has very high clinical guiding significance.

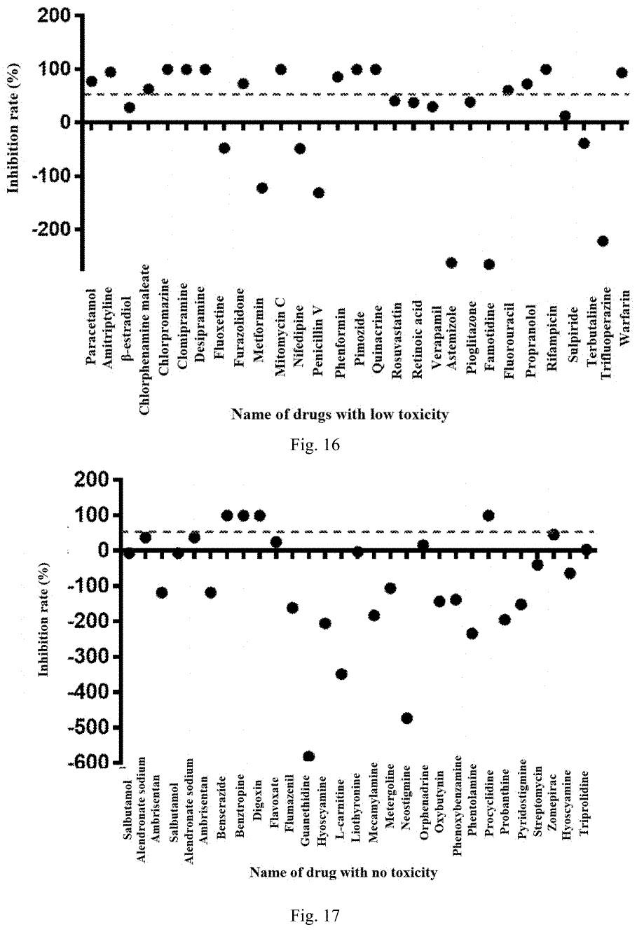

In the embodiments of the disclosure, a large-scale hepatotoxicity test of more than 100 marketed drugs is carried out on the 3D human liver organ model, wherein drug types include reported clinically types with severe hepatotoxicity, high hepatotoxicity, low hepatotoxicity and no hepatotoxicity, so as to illustrate the high predictability of the 3D human liver organ model in the embodiments of the disclosure for the hepatotoxicity of drugs. Wherein, as shown in FIG. 16 and FIG. 17, representative results of screening drugs with low hepatotoxicity and no hepatotoxicity in the large-scale drug screening of the 3D human liver organ model II-2 of this embodiment 2 are shown respectively. Wherein, the ordinate in the figure is an inhibition rate, which can reflect the in vitro hepatotoxicity of drugs. The greater the inhibition rate, the higher the toxicity. It can be seen from FIGS. 16-17 that a clinical prediction rate of the 3D human liver organ model II-2 is very high, and prediction results of hepatotoxicity of various types of drugs are highly consistent with the clinical conclusions. Thus, the 3D human liver organ model II-2 can be used to predict the hepatotoxicity of unknown drugs in vitro, and has high clinical guiding significance. Wherein, the classification of high hepatotoxicity, low hepatotoxicity and no hepatotoxicity is the same as that in clinical practice.

In the embodiments of the disclosure, the large-scale drug screening results of the 3D human liver organ model II-2 are analyzed and counted, and the drug screening results are divided by taking a drug inhibition rate and IC50/Cmax=50 as critical criteria for hepatotoxicity. It is concluded that the sensitivity of the model is 63.2%, and the prediction rate of overall hepatotoxic drugs is 70.7%, which are higher than other cell models and animal screening results reported.

Some comparative examples are provided below for comparative analysis with the embodiments in the embodiments of the present disclosure.

### Comparative Example 1

A 2D liver organ model constructing method, including the following steps:

S11′, digesting 2D cultured human liver cancer cell lines hepG2 in a growth period into a single cell suspension by using 0.25 (vt.)% pancreatin, and carrying out centrifugation and resuspension to obtain a single cell suspension with a density of (0.18-1.34)×10′ cell/mL.

S21′, mixing the single cell suspension with a culture matrix according to a volume ratio of the single cell suspension to the culture matrix being 5.6:94.4 to obtain a mixed cell suspension; wherein a 1640 culture medium is adopted as the culture matrix.

S31′, inoculating a 96-well plate (100 μL/well) with the mixed cell suspension, wherein the number of inoculated cells in each well is 2000-7500; and carrying out cultivation at 37° C. to obtain a 2D human liver organ model.

A 2D human liver organ comparison model I is constructed in comparative example 1. Wherein different 2D human liver organ comparison models I are constructed according to different numbers of inoculated cells in each cultivation micropore in the step S31′. A 2D human liver organ comparison model I-1 is constructed according to the fact that the number of cells in each cultivation micropore is 2000. A 2D human liver organ comparison model I-2 is constructed according to the fact that the number of cells in each cultivation micropore is 5000. A 2D human liver organ comparison model I-3 is constructed according to the fact that the number of cells in each cultivation micropore is 7500.

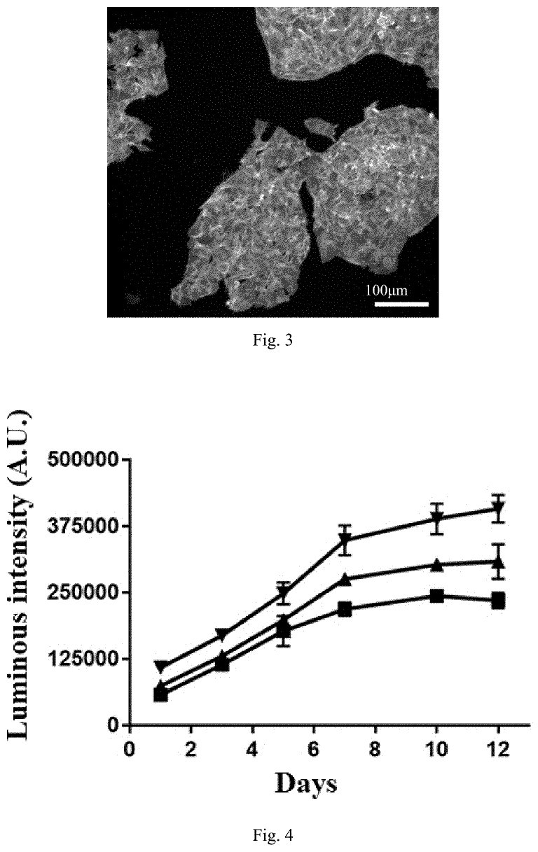

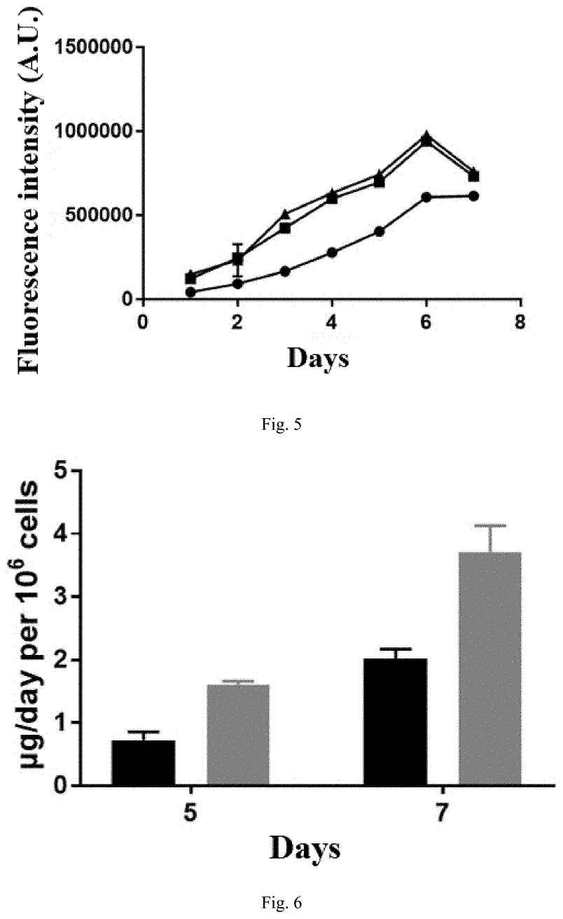

In this comparative example 1, F-actin and nuclear dyeing characterization is carried out on the 2D human liver organ comparison model I. As shown in a cell morphology diagram of a 2D human liver organ comparison model I-1 in FIG. 3, it can be seen that liver cells in the 2D liver organ comparison model I grow in a single-layer spreading adherent manner. As shown in a cell growth characterization curve graph of a 2D human liver organ comparison model I in FIG. 5, it can be seen that the cells can only grow for 6 days at most and the vitality of the cells will enter a decline state on the 2D human liver organ comparison model I. Wherein in FIG. 5, “-●-” is a cell growth curve of the 2D comparison model I-1, “-▪-” is a cell growth curve of the 2D comparison model I-2, and “-▴-” is a cell growth curve of the 2D comparison model I-3.

### Comparative Example 2

A 2D liver organ model constructing method, including the following steps:

S12′, resuscitating human primary liver cells preserved by liquid nitrogen, digesting the human primary liver cells into a single cell suspension, and carrying out centrifugation and resuspension to obtain a single cell suspension with a density of 1.78×10⁶ cell/mL.

S22′ mixing the single cell suspension with a culture matrix according to a volume ratio of the single cell suspension to the culture matrix being 50:50 to obtain a mixed cell suspension; wherein a matched primary liver cell culture medium is adopted as the culture matrix.

S32′, inoculating a 96-well plate with the mixed cell suspension, wherein the number of inoculated cells in each well is 50000; and then carrying out cultivation at 37° C. to obtain a 2D primary liver model.

A 2D human liver organ comparison model II is constructed in comparative example 2.

In this comparative example 2, F-actin and nuclear dyeing characterization is carried out on the 2D human liver organ comparison model H. As shown in a cell morphology diagram of a 2D human liver organ comparison model II in FIG. 11, it can be seen that liver cells in the 2D human liver organ comparison model II grow in a single-layer spreading adherent manner.

### Comparative Example 3

A 3D human liver organ comparison model constructing method, including the following steps:

S13′, resuscitating human primary liver cells preserved by liquid nitrogen, digesting the human primary liver cells into a single cell suspension, and carrying out centrifugation and resuspension to obtain a single cell suspension with a density of 7.14×10⁶ cell/mL.

S23′, adding the single cell suspension and a matrix material into a 1.5 ml EP tube according to a volume ratio of the single cell suspension to the matrix material being 5.6:2.4, and conducting uniform mixing through pipetting by a pipette to obtain a mixed cell suspension; wherein the matrix material is a mixed matrix material of collagen and matrigel, a volume ratio of the collagen to the matrigel is 1:1, the concentration of the collagen is 2.5 mg/mL, and the concentration of the matrigel is 5 mg/mL.

S33′, quickly transferring the mixed cell suspension at high throughput by using a multichannel pipette to inoculate cultivation micropores of a 3D organ-on-a-chip, then placing the 3D organ-on-a-chip inoculated with the mixed cell suspension in a cell incubator, carrying out cultivation at 37° C. for 10 min while ensuring that collagen can be gelled well, and taking out the 3D organ-on-a-chip to obtain a gelled 3D organ-on-a-chip; wherein the number of cells in each cultivation micropore is 35000-40000;

S43′, adding a culture medium into liquid storage holes of the gelled 3D organ-on-a-chip, and then adding a PBS buffer solution into an anti-liquid-evaporation channel of the gelled 3D organ-on-a-chip. And carrying out cultivation at 37° C. to obtain a 3D human liver organ model.

A 3D human liver organ comparison model III is constructed in comparative example 3.

In this comparative example 3, F-actin and nuclear dyeing characterization is carried out on the 3D human liver organ comparison model I. As shown in a cell morphology diagram of a 3D human liver organ comparison model III in FIG. 12, it can be seen that the pseudopodia of cells is almost invisible, and the bionic performance becomes poor.

In the embodiments of the disclosure, the human primary liver cells in Embodiment 2 can be replaced by mixed cells of the human primary liver cells and the liver non-parenchymal cells, and various detection results of the obtained 3D human liver organ model are similar to those in Embodiment 2 and are not repeated here.

In the embodiments of the disclosure, detection on the liver organ model is carried out by adopting the following technical means:

1. Characterization of the cell proliferation capacity in a liver model: the vitality and proliferation state of the cells are subjected to characterization by using a cell ATP characterization kit Cell titer 3D glo, liver models constructed in a same batch are detected and quantified every day, and characterization is continuously performed for 7 days.

2. Quantification of the liver function: quantitative characterization is carried out on mRNA expressed by albumin and metabolic enzymes in the liver function of the constructed 2D and 3D human liver organ models by adopting a commercial kit.

3. Screening of hepatotoxic drugs: 15 hepatotoxic drugs to be tested dissolved in DMSO in advance are prepared by using a proper culture medium according to the test concentration, the culture medium in the constructed 3D liver cell line organ model cultured for 4 days and the constructed 2D liver cell line organ model cultured for 24 hours is removed, 100 μL of a cell culture medium containing 100 μM of hepatotoxic drugs is added, the 2D organ model is continuously cultured for 24-72 h, and the 3D hepatotoxicity organ model is continuously cultured for 3-8 days. In a long-time administration test of the 3D human liver organ model, 100 μM is administered once, and if repeated administration is required, a drug is changed in the middle and the concentration is maintained. Each 3D human liver organ model and each 2D liver organ model need to be provided with a blank group, a negative control group and a positive control group, and each group is provided with six replicate wells.

4. Drug sensitivity result detection: the 3D hepatotoxic drug screening system can be subjected to characterization by using the existing drug sensitivity detection means, and a cell metabolism capability evaluation system, if cell titer blue is added, compares the cell metabolic capability and evaluates the effect of drugs. After drug incubation is completed, the culture medium with the drugs is removed, 100 μL of a mixed solution of a cell titer blue stock solution and a complete culture medium in a ratio of 1:5 is added, incubation is conducted for 3 h at 37° C., and a detection wavelength is 560 em/590 ex nm. For example, the Cell titer 3D glo is added to evaluate the ATP of the 3D liver model, so that the action effect of the drugs is evaluated. After drug incubation is completed, the culture medium with the drugs is removed, 100 μL of a mixed solution of a Cell titer 3D glo stock solution and a complete culture medium in a ratio of 1:1 is added, incubation is conducted for 0.5 h at normal temperature, and detection is conducted in a Luminescence mode. For example, a high-content imaging technology is used for imaging characterization of the number of living and dead cells, and characterization of cell mitochondrial activity and membrane potential to evaluate hepatotoxicity drugs. The embodiment is applicable to, but not limited to, the above characterization method.

In the embodiments of the disclosure, the liver organ chip drug screening system can be used for characterizing the vitality of liver cells after drug action by using the existing ATP and metabolic capability detection means, can also be used for characterizing the cell mitochondrial function by using a mitochondrial membrane potential kit or a mitochondrial active oxygen free radical kit, and can also be used for characterizing the liver function of the model by using a liver function characterization kit such as albumin and a-GST. Therefore, the hepatotoxic effect results of the drugs may be subjected to characterization by using a single parameter or multiple parameters.

The above description and drawings sufficiently show embodiments of the present disclosure to enable those skilled in the art to practice the embodiments. Other embodiments may include structural and other changes. Embodiments represent only possible changes. Unless explicitly required, Individual components and functions are optional, and the order of operation may vary. Portions and features of some embodiments may be included in or replace portions and features of other embodiments. Embodiments of the present disclosure are not limited to the structures that have been described above and shown in the drawings, and various modifications and changes can be made without departing from the scope of the disclosure. The scope of the present disclosure is limited only by the appended claims.

1. A 3D human liver organ model constructing method, comprising:
preparing human primary liver cells, or mixed cells of the human primary liver cells and liver non-parenchymal cells, or human liver cancer cell lines into a single cell suspension;
mixing the single cell suspension with a matrix material to obtain a mixed cell suspension;
inoculating cultivation micropores of a 3D organ-on-a-chip with the mixed cell suspension, and carrying out cultivation at 37° C. to obtain a gelled 3D organ-on-a-chip; wherein in each cultivation micropore, an inoculation volume of the mixed cell suspension is 6-10 μL, the number of inoculated cells of the human primary liver cells is 2500-25000, and the number of inoculated cells of the human liver cancer cell lines is 500-10000; and
adding a culture medium into liquid storage holes of the gelled 3D organ-on-a-chip, and carrying out cultivation at 37° C. to obtain a 3D human liver organ model.

2. The 3D human liver organ model constructing method according to claim 1, further comprising:
digesting human primary liver cells into a single cell suspension, and carrying out centrifugation and resuspension to obtain a single cell suspension with a density of (4.45-44.5)×10⁶ cell/mL.

3. The 3D human liver organ model constructing method according to claim 2, further comprising:
resuscitating and diluting human primary liver cells into a single cell suspension, and carrying out centrifugation and resuspension to obtain a single cell suspension with a density of 8.9×10⁶ cell/mL.

4. The 3D human liver organ model constructing method according to claim 2, wherein,
the number of inoculated cells of the human primary liver cells is 5000-15000.

5. The 3D human liver organ model constructing method according to claim 1, further comprising:
digesting human liver cancer cell lines hepG2 into a single cell suspension, and carrying out centrifugation and resuspension to obtain a single cell suspension with a density of (0.89-8.9)×10⁶ cell/mL.

6. The 3D human liver organ model constructing method according to claim 5, further comprising:
digesting human liver cancer cell lines hepG2 into a single cell suspension, and carrying out centrifugation and resuspension to obtain a single cell suspension with a density of 3.56×10⁶ cell/mL.

7. The 3D human liver organ model constructing method according to claim 5, wherein,
the number of inoculated cells of the human liver cancer cell lines is 500-3000.

8. The 3D human liver organ model constructing method according to claim 1, wherein after adding the culture medium into the liquid storage holes of the gelled 3D organ-on-a-chip, the method also comprises:
adding a buffer solution into an anti-evaporation structure of the gelled 3D organ-on-a-chip.

9. A 3D human liver organ model, characterized by being constructed by adopting the constructing method according to claim 1.

10. Use of the 3D human liver organ model according to claim 9 for screening of hepatotoxic drugs.
